(19) United States
(12) Patent Application Publication
(10) Pub. No.: US 2019/0142247 A1
(43) Pub. Date: May 16, 2019

(54) Endoscope and Treatment Tool Drive Module

(71) Applicants: MakeWay LLC, Saitama (JP); PMT Corporation, Fukuoka (JP); Taira Denki Co., Ltd., Shizuoka (JP)

(72) Inventors: Masanori Maeda, Saitama (JP); Akihiro Ikenaga, Fukuoka (JP); Masami Yoshino, Fukuoka (JP)

(21) Appl. No.: 16/304,424

(22) PCT Filed: May 25, 2017
(86) PCT No.: PCT/JP2017/019627

§ 371 Date: Nov. 26, 2018

(30) Foreign Application Priority Data
May 26, 2016 (JP) 2016-105626

(51) Int. Cl. A61B 1/00; A61B 1/018

(52) U.S. Cl. CPC A61B 1/00133; A61B 1/018

(57) ABSTRACT

An endoscope according to the present invention comprises: a tubular insertion portion 2 to be inserted into a living body; and a treatment tool channel formed inside the insertion portion 2, the treatment tool channel being configured to guide a treatment tool 51 by passing the treatment tool 51 through the treatment tool channel, wherein the endoscope comprises: a treatment tool driver 7 for allowing a forward movement and/or a backward movement of the treatment tool 51 in the treatment tool channel; and a drive controller 8 for controlling the forward movement and/or the backward movement of the treatment tool 51 by the treatment driver 7.

### TECHNICAL FIELD

The present invention relates to an endoscope comprising a tubular insertion portion to be inserted into a living body and a treatment tool channel inside the insertion portion, the treatment tool channel guiding a treatment tool therethrough. More particularly, the present invention proposes a technique that can dramatically improve convenience for use of a treatment tool, particularly during a surgical operation using an endoscope.

### BACKGROUND ART

An endoscope is widely used since long ago. The endoscope can be inserted, for example, from a mouth, nasal cavity, anus or body surface skin into a hollow organ such as a digestive tract, a trachea and a blood vessel or a solid organ in a living body, and can observe the inside of the living body from the outside by means of an optical system built in the endoscope while enabling a required treatment such as a therapeutic. In accordance with recent enhancement of an optical technology, an imaging technology or the like, various improvements have been added to the endoscope, and the endoscope has been developed.

Specifically, the endoscope includes various types such as an upper gastrointestinal endoscope, a duodenum endoscope, a colon endoscope and the like, and they are selectively used depending on positions for use and the like in the living body. In general, except for special endoscopes such as capsule endoscopes, any of the endoscopes mainly includes: an insertion portion extending in an elongated shape and being inserted into the living body; and an operation portion disposed on a base end side of the insertion portion, in which a user grips the operating portion and performs a predetermined operation.

The operation potion of the endoscope is provided with an angle knob or the like for the user to operate a bending operation of the insertion portion in up and down and right and left directions when the insertion portion is inserted into the living body, and the operation portion may be connected to a video scope that displays an image obtained from an imaging element provided at a tip portion of the insertion portion.

The insertion portion of the endoscope has a tubular shape having an outer diameter that can be inserted into the living body and having a portion capable of performing the bending operation based on the user's operation at the operation portion. In the inner portion of the insertion portion, a plurality of channels are generally formed, including a treatment tool channel for passing a forceps, a snare or other treatment tool through the channel to guide the tool to the distal end portion.

### SUMMARY OF INVENTION

### Technical Problem

When using the endoscope as described above, the user grips the operation portion by one hand (mainly a left hand) to perform a required operation and also performs a predetermined treatment by the other hand (mainly a right hand) while grasping and supporting a front portion of the insertion portion which is to be inserted into the living body. Therefore, under such a circumstance, both hands of the user are occupied.

Under this circumstance, the user frequently adjusts the state of delivery of the treatment tool from the treatment tool channel of the endoscope during the treatment. In this case, a forward movement and backward movement of the treatment tool inserted into the treatment tool channel is performed by manually moving and manipulating the treatment tool from a treatment tool insertion port side on the base end side of the insertion portion. Therefore, when attempting to move the treatment tool forward or backward, the hand holding the insertion portion of the endoscope is temporarily released and the treatment tool is moved with that hand, or if it is difficult to release the hand holding the endoscope, an assistant different from the user moves the treatment tool forward or backward.

However, in order to accurately display a lesioned part on the image, the user of the endoscope manipulates the endoscope while adjusting a slight pushing state and a twisting state of the insertion portion by the hand holding the insertion portion. Therefore, in the first place, it is not desirable to release the holding portion held for moving the treatment tool. Also, when the treatment tool is to be moved by the assistant, the forward and backward movements of the treatment tool to a desired position often takes much time due to a communication gap between the assistant and the user. In the first place, there are cases where no assistant is available. Therefore, the conventional endoscope has had poor convenience.

Further, while inserting the insertion portion into the living body, the treatment tool inserted into the treatment tool channel may be replaced with another treatment tool. In this case, the assistant different from the user or the user will pull out and remove the treatment tool having a length of several meters in the treatment tool channel, and also will insert another treatment tool having a length of several meters into the treatment tool channel. Therefore, in the conventional endoscope, the replacement of the treatment tool has been a troublesome work requiring much time and labor.

An object of the present invention is to solve such problems of the conventional endoscopes. The object is to provide an endoscope and a treatment tool drive module, which can dramatically improving convenience for use in a treatment tool when performing a treatment using an endoscope.

### Solution to Problem

An endoscope according to the present invention comprises: a tubular insertion portion to be inserted into a living body; and one or more treatment tool channels formed inside the insertion portion, the treatment tool channels being configured to guide a treatment tool by passing the treatment tool through the treatment tool channel, wherein the endoscope comprises: a treatment tool driver for allowing a forward movement and/or a backward movement of the treatment tool in the treatment tool channel; and a drive controller for controlling the forward movement and/or the backward movement of the treatment tool driven by the treatment driver.

Here, the endoscope according to the present invention comprises an endoscope body including the insertion portion, and the treatment tool driver comprises a treatment tool drive module attachable to and detachable from the endoscope body.

Alternatively, the endoscope according to the present invention comprises an endoscope body including the insertion portion, and the treatment tool driver is integrally incorporated in the endoscope body.

In the endoscope according to the present invention, the treatment tool driver comprises: one or more rotating wheels that are brought into contact with the treatment tool and allow the forward movement and/or backward movement of the treatment tool, preferably a pair of rotating wheels that are rotated in opposite directions and allow the forward movement and/or backward movement of the treatment tool sandwiched between the rotating wheels; and an electric motor or a pneumatic motor (a turbine) or other various types of motors or actuators, for driving the rotating wheels to rotate them by electric power or air supply.

In this case, it is more preferable that the treatment tool driver further comprises a wheel housing for containing the rotating wheels, wherein the motor is attached to the wheel housing and at least a part of the wheel housing is destroyed by removing the motor.

Further, in the endoscope according to the present invention, the drive controller preferably comprises: an input portion for receiving an input from the outside; and an output portion for transmitting a signal corresponding to the input received by the input portion to the treatment tool driver.

In this case, the input portion of the drive controller is configured to be capable of receiving a plurality of types of inputs, and the output portion is configured to generate signals having different movement speeds of the forward movement and/or the backward movement of the treatment tool driven by the treatment tool driver, in response to the input received by the input portion.

Furthermore, in the endoscope according to the present invention, it is preferable that the drive controller is a foot pedal manipulated by a user.

The endoscope according to the present invention preferably comprises a sensor capable of detecting a marking made in a predetermined position of the treatment tool.

A treatment tool drive module according to the present invention is used for an endoscope comprising an endoscope body including a tubular insertion portion to be inserted into a living body, and is attachable to and detachable from the endoscope body, and the treatment tool drive module allows a forward movement and/or a backward movement of the treatment tool in a treatment tool channel formed inside the insertion portion of the endoscope body.

The treatment tool drive module according to the present invention preferably comprises: one or more rotating wheels that are brought into contact with the treatment tool and allow the forward movement and/or backward movement of the treatment tool, preferably a pair of rotating wheels that are rotated in opposite directions and allow the forward movement and/or backward movement of the treatment tool sandwiched between the rotating wheels; and a motor for driving the rotating wheels to rotate them.

In this case, it is more preferable that the treatment tool drive module further comprises a wheel housing for containing the rotating wheels, wherein the motor is attached to the wheel housing and at least a part of the wheel housing is destroyed by removing the motor.

The treatment tool drive module according to the present invention preferably comprises a sensor capable of detecting a marking made in a predetermined position of the treatment tool.

### Advantageous Effects of Invention

According to the endoscope of the present invention, the endoscope includes the treatment tool driver for allowing the forward movement and/or the backward movement of a treatment tool in the treatment tool channel and the drive controller for controlling the forward movement and/or the backward movement of the treatment tool by the treatment tool driver, thereby enabling easy forward and/or backward movement of the treatment tool in the treatment tool channel. Therefore, the endoscope according to the present invention can dramatically improve convenience for use of the treatment tool when performing the surgical treatment using the endoscope.

When the treatment tool driver is constituted by a treatment tool drive module which can be attached to and detached from the endoscope body, the treatment tool can be easily moved forward and backward in the treatment tool channel only by attaching the treatment tool drive module to the existing endoscope, so that it is possible to very easily obtain the above effect of improving convenience.

Further, the treatment tool drive module through which the treatment tool passes may be required to be washed every time of use. However, the present invention also provides an advantage that the washing becomes easy, because the treatment tool drive module is attachable to and detachable from the endoscope. Alternatively, the treatment tool drive module can be disposable.

When the above treatment tool driver is integrally incorporated in the endoscope body, a work for attaching and detaching the module can be eliminated to improve further convenience, as compared with a case where the treatment tool drive module is used. In addition, the incorporating of the endoscope in an appropriate position of the endoscope body can allow suppression of an increase in size of the entire endoscope.

When the treatment tool driver includes a pair of rotating wheels which rotate in opposite directions to each other to allow the forward movement and/or the backward movement of the treatment tool sandwiched therebetween; and a motor which rotationally drives the rotating wheels, the forward movement and/or the backward movement can be achieved with a simple structure.

When the drive controller includes the input portion for receiving the input from the outside; and the output portion for transmitting the signal corresponding to the input received by the input portion to the treatment tool driver, the forward movement and/or the backward movement of the treatment tool can be surely performed based on the intention of the user or the like from the outside.

In this case, when the input portion of the drive controller is configured to be capable of receiving a plurality of types of inputs, and the output portion is configured to generate signals having different movement speeds and movement distances of the forward movement and/or the backward movement of the treatment tool by the treatment tool driver, in response to the input received by the input portion, the movement speed of the forward movement or the backward movement of the treatment tool can be changed by the intention of the user or the like, so that a treatment with higher accuracy can be carried out.

When the above drive controller is the foot pedal manipulated by the foot of the user, for example, the user gripping the operation portion and the insertion portion with both hands can effectively utilized his/her foot that can be freely moved, when using the endoscope, for example.

When the endoscope according to the present invention includes a sensor capable of detecting a marking to be made in a predetermined position of the treatment tool, sudden protrusion or the like of the treatment tool from the distal end of the insertion portion of the endoscope can be prevented, thereby improving the safety and eliminating concerns of the user for such protrusion or the like, for example when inserting the treatment tool at a relatively high speed in replacing the treatment tool or the like.

The treatment tool drive module of the present invention can be attached to and detached from the endoscope body and can allow the forward movement and/or the backward movement of the treatment tool in the treatment tool channel formed inside the insertion portion of the endoscope, whereby the treatment tool drive module can be used for an existing endoscope, can significantly improve convenience for the existing endoscope, and can be easily washed by removing it from the endoscope body after use. Alternatively, all or a part of the treatment tool drive module can be disposable to discard after being used about once.

When the treatment tool driver module includes a pair of rotating wheels that rotate in opposite directions to each other to allow the forward movement and/or the backward movement of the treatment tool sandwiched therebetween, and an electric motor that rotationally drives the rotating wheels, the forward movement and/or the backward movement of the treatment tool can be achieved with a simple structure.

When the treatment tool drive module according to the present invention includes a sensor capable of detecting a marking to be made in a predetermined position of the treatment tool, it can contribute to the improvement of safety and elimination of concerns of the user, as described above.

### BRIEF DESCRIPTION OF DRAWINGS

FIG. 1 is a perspective view showing an endoscope according to one embodiment of the present invention.

FIG. 2 is a perspective view showing an endoscope treatment tool driver in FIG. 1 with an endoscope body removed.

FIG. 3 is a front view showing a drive wheel portion of the treatment tool driver of FIG. 2 in a state where a wheel housing is opened, and a cross-sectional view taken along the line b-b of the drive wheel portion.

FIG. 4 is a front view showing a motor portion of the treatment tool driver in FIG. 2 in a state where a motor case is opened.

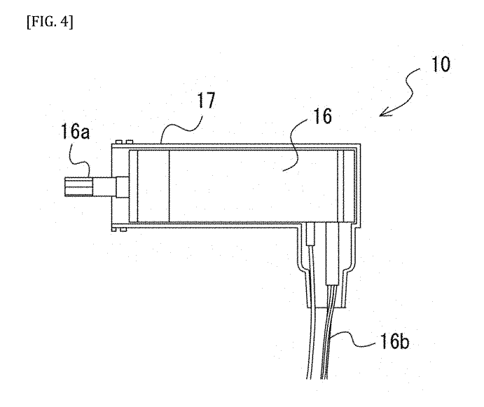

FIG. 5 is an exploded perspective view showing an endoscope connecting portion of the treatment tool driver of FIG. 2.

FIG. 6 is an enlarged perspective view of a treatment tool inserting port of the endoscope of FIG. 1.

FIG. 7 is a perspective view showing a variation of a wheel housing of a treatment tool driver.

FIG. 8 is an exploded plan view of each component of the wheel housing of FIG. 7.

FIG. 9 is a front view of the wheel housing of FIG. 7.

FIG. 10 is a bottom view of the wheel housing of FIG. 7.

FIG. 11 is a bottom view showing the wheel housing of FIG. 7 with an electric motor attached.

FIG. 12 is a perspective view showing a drive controller of the endoscope of FIG. 1.

FIG. 13 is a plan view of the drive controller of FIG. 12.

FIG. 14 is a perspective view showing a main part of an endoscope according to another embodiment of the present invention.

FIG. 15 is a partial cross-sectional view of the endoscope of FIG. 14.

FIG. 16 is a partial cross-sectional perspective view of the endoscope of FIG. 14.

### DESCRIPTION OF THE PREFERRED EMBODIMENTS

Embodiments of the present invention will be described in detail below with reference to the drawings.

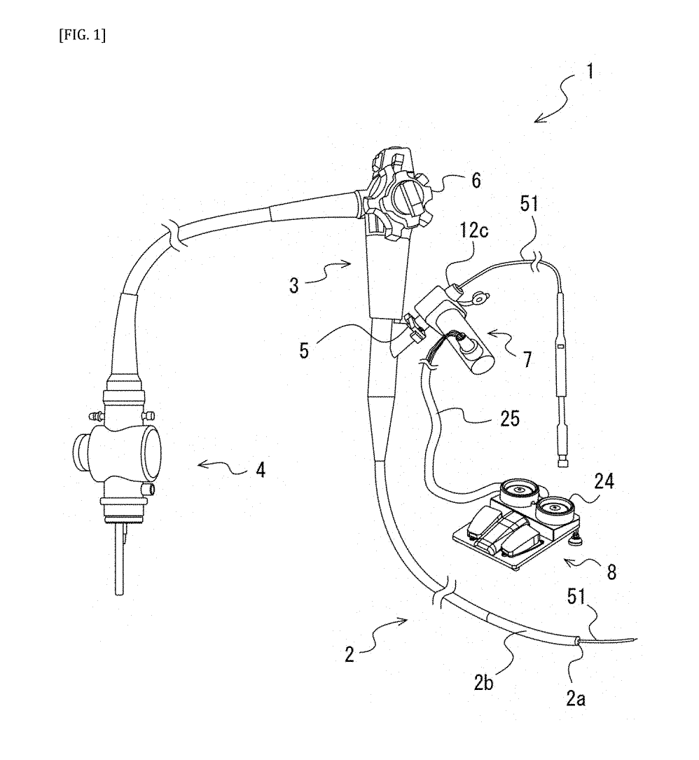

An endoscope 1 according to one embodiment of the present invention is mainly used for a medical practice. As illustrated in FIG. 1, the endoscope 1 includes: a tubular insertion portion 2 to be inserted into a living body, particularly a human body; and an operation portion 3 provided on a base end side of an insertion portion 2 and for performing, by a user, a required operation such as a bending operation of the insertion portion 2 and insertion of a treatment tool described below.

The endoscope 1 shown is of a video scope type having an imaging element (CCD) (not shown) mounted on a distal end portion 2a of the insertion portion 2, and is provided with a connecting portion 4 used for connection to a monitor for displaying an image from the imaging element, the connecting portion 4 extending from the operating portion 3.

Here, in the insertion portion 2, one or more treatment tool channels through which a treatment tool 51 are passed inside the full length of the insertion portion. Thus, as shown in FIG. 1, the distal end portion of the treatment tool 51 can be inserted from a treatment tool insertion port 5 provided on the base end side of the insertion portion 2, pass through the treatment tool channel, and protruded from a treatment tool protruding port at the distal end portion 2a of the insertion portion 2.

For the treatment tool 51, an existing treatment tool or the like can be used. Specifically, for example, the treatment tool 51 can have a shape such as a tubular shape having a small outer diameter sufficient to pass through the treatment tool channel, and can be provided with a biopsy forceps, a forceps with a needle, a standard forceps, a grasping type forceps, a hot biopsy forceps, a forceps with hemostatic clip and/or a high frequency hemostatic forceps, or a cell brush, a liquid spray or infusion tube, a high frequency snare, a transparent cap, a acicular scalpel, a needle knife, a high frequency knife or the like.

Further, in the embodiment shown, the operation portion 3 includes a plurality of angle knobs 6 connected to the insertion portion 2 by wires or the like. The operating of these angle knobs 6 can cause a bending movement of a curved portion 2b provided on the distal end side in up and down or right and left directions. It should be noted that a portion other than the curved portion 2b of the insertion portion 2 can be generally soft enough to follow a shape of a lumen or the like into which the treatment tool is inserted.

In addition, a suction button or an air/water supply button can be provided on the operation unit 3 as necessary. In this case, after the distal end portion 2a of the insertion portion 2 reaches a predetermined position in the living body, with these buttons, it is possible to allow suction or release of a liquid or gas from the distal end portion 2a in the living body.

Further, the operation portion 3 may be optionally provided with a suction button or an air/water supply button. In this case, after the distal end portion 2a of the insertion portion 2 reaches a predetermined position in the living body, these buttons allow suction or release of a liquid or a gas from the distal end portion 2a at that position in the living body.

In addition, the structures of the insertion portion 2 and the operation portion 3 forming the endoscope body are substantially the same as those of the existing endoscope, and for example, an objective lens and a light guide may be provided at the distal end portion 2a of the insertion portion 2, and an ocular lens or a diopter adjusting ring may be provided at the operation portion 3, as needed.

Here, in the embodiment, the treatment tool driver 7 for allow the forward movement and the backward movement of the treatment tool 51 in the treatment tool channel is attached to the treatment tool insertion port 5 located between the operation portion 3 and the insertion portion 2, and a drive controller 8 for controlling the forward movement and the backward movement of the treatment tool 51 by the treatment tool driver is provided. It is to be noted that the treatment tool driver 7 is included in the present invention as long as it can cause at least one of the forward movement and the backward movement of the treatment tool 51. Here, both of the forward and backward movements can be generated.

Here, after passing through the inside of the treatment tool driver 7, the treatment tool 51 is inserted into the treatment tool channel from the treatment tool insertion port 5 of the endoscope body.

According to this, under the control of the drive controller 8, the treatment tool 51 can be moved forward or moved backward within the treatment tool channel by the operation of the treatment tool driver 7, so that it is possible to greatly increase the convenience for use of the treatment tool 51 and thus the easiness of performing the treatment procedure using the endoscope 1, as compared with the conventional endoscope in which the treatment tool was manually moved forward and backward.

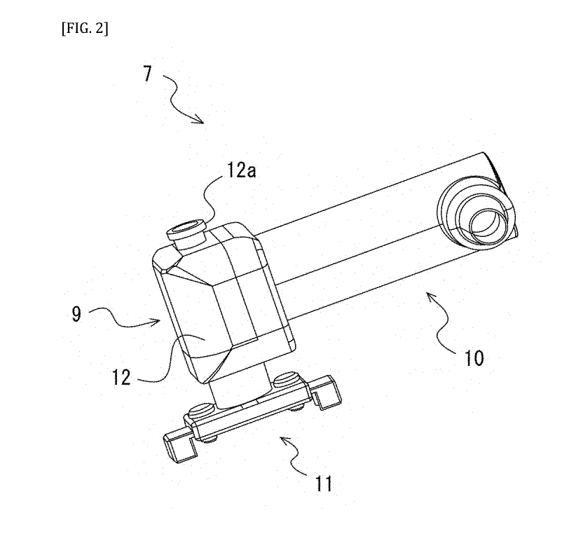

The treatment tool driver 7 will be described in detail. In the embodiment shown in FIG. 1, the treatment tool driver 7 is in the form of a treatment tool drive module as a part attachable to and detachable from the treatment tool insertion port 5 of the endoscope body, as shown in FIG. 2.

For example, the treatment tool driver 7 includes: a drive wheel portion 9 for driving the treatment tool 51 forward and backward; a motor portion 10 for applying a driving force to the drive wheel portion 9 based on supply of electric power from a power source (not shown); and an endoscope connecting portion 11 for attaching the treatment tool driver 7 to the endoscope body.

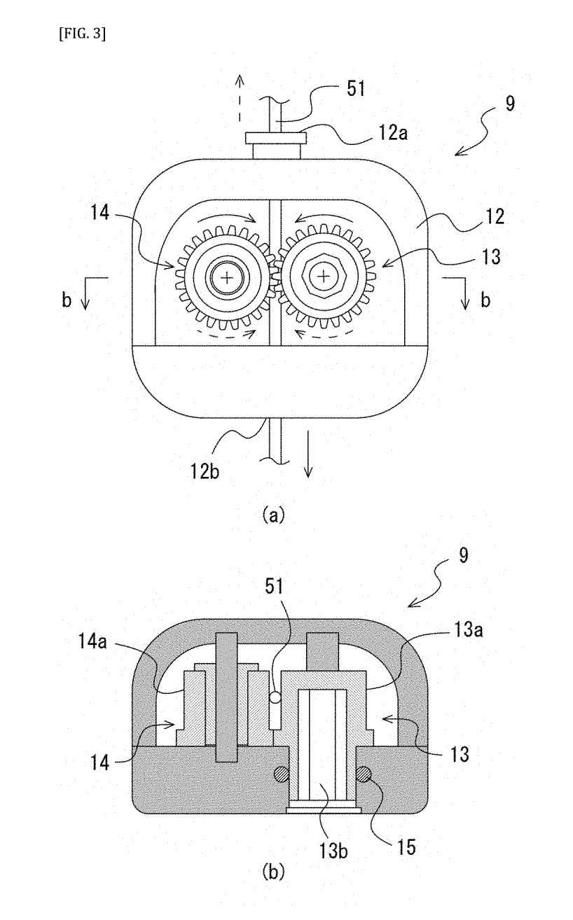

Here, as shown in FIGS. 3(a) and 3(b), the drive wheel portion 9 of the treatment tool driver 7 is configured such that a pair of rotating wheels 13, 14 each having a gear is rotatably supported and housed in a wheel housing 12 having a treatment tool inlet 12a and a treatment tool outlet 12b. As shown in FIG. 1, an insertion port cap 12c can be attached to the treatment tool inlet 12a, and the treatment tool 51 is inserted into the wheel housing 12 via the insertion port cap 12c.

Specifically, the pair of rotating wheels 13, 14 includes a driving side wheel 13 connected to and rotatively driven by an electric motor 16 (as described below) of the motor portion 10; and a driven side wheel 14 having a gear engaged with the gear of the driving side wheel 13 and rotated by power transmission from the driving side wheel 13. It should be noted that the connecting portion to the electric motor 16 in the driving side wheel 13 can be, for example, a concave portion 13b having an octagon or other polygonal hole shape as shown in FIG. 3(b), so as to correspond to a shape of a shaft 16a of the electric motor 16.

As can be seen from FIG. 3(b), both of these rotating wheels 13, 14, in this embodiment, are provided with rollers 13a, 13b, which each has a smooth outer surface in contact with the treatment tool and is coaxial with the gear.

In the embodiment shown, the outer surface of each of the rollers 13a, 14a has a smooth curved surface. However, the outer surface of each of the rollers may have an uneven surface with a predetermined pattern such as groove or depression, although not shown. In this case, depending on such a pattern, there is an advantage that it is possible to repel a liquid which will otherwise cause idling.

With such a configuration of the drive wheel portion 9, when a driving force is applied from the motor portion 10 to the driving side wheel 13, the driving side wheel 13 and the driven side wheels 14 are rotated in opposite directions to each other as shown by a solid arrow in FIG. 3a, thereby allowing the forward movement of the treatment tool 51 sandwiched between the rollers 13a, 14a of the rotating wheels 13, 14 in a direction of extending to a direction adjacent to the rotating wheels 13, 14 and in a direction substantially orthogonal to the axial direction.

By rotating the electric motor 16 in the reverse direction, the rotational directions of the rotating wheels 13, 14 are also reversed, as indicated by the broken line arrows in FIG. 3a. In this case, the treatment tool 51 can be moved backward.

It is preferable to provide a lining layer made of polyurethane, that is, an urethane rubber or other rubber or resin on the outer surface of each of the rollers 13a, 14a of the rotating wheels 13, 14, whereby when the treatment tool 51 is moved forward and backward, a required frictional force can be ensured between the rollers and the treatment tool 51 to prevent the treatment tool 51 from slipping. In particular, a distance between the rollers 13a, 14a herein may preferably be, for example, from 0.7 mm to 1.2 mm, and more preferably from 1.6 mm to 3.2 mm, as measured along the direction orthogonal to the rotational axis of the rotating wheels 13, 14. This can be addressed with the treatment tool 51 including those having various thicknesses, so that various treatment tools 51 can be effectively moved forward and backward, between the pair of rotation wheels 13, 14 with elastic deformation of the lining layer as described above.

In order to prevent a liquid adhering to the treatment tool 51 from entering the motor portion 10 from the inside of the wheel housing 12 in association with use, a sealing material such as an O-ring may be preferably provided at a connecting portion to the electric motor in the driving side wheel 13.

When inserting the treatment tool 51 from the treatment tool inlet 12a of the wheel housing 12, the treatment tool 51 is manually forced into a position where a distal end portion of the treatment tool 51 is brought into contact with the rollers 13a, 14a, for example, into a position of about 20 mm from the treatment tool inlet 12a. After the distal end portion of the treatment tool 51 is brought into contact with the rollers 13a, 14a, the treatment tool 51 can be moved forward by the rotationally driven rollers 13a, 14a. By thus providing a certain distance between the treatment tool inlet 12a and the rollers 13a, 14a, the treatment tool 51 will be held without being detached from the insertion port cap 12c attached to the treatment tool inlet 12a even if the treatment tool 51 is moved backward at a high speed when taking out the treatment tool 51 after use. This configuration is useful for preventing the distal end portion of the treatment tool 51 polluted by contact with a diseased part of a patient from uncontrollably jumping out to the outside of the endoscope 1.

It is desirable that the drive wheel portion 9 is disposable to be discarded after use, for example, after being used about once. However, in this embodiment, since the drive wheel portion 9 is configured to be detached from the treatment tool driver 7, the driving while portion 9 may be easily cleaned and reused.

Here, the treatment tool driver 7 has a motor portion 10 at a position adjacent to the drive wheel portion 9 in order to apply a driving force to the driving side wheel 13 of the above drive wheel portion 9.

As with the existing motor, the motor portion 10 is provided with an electric motor 16 having a rotating shaft 16a and a lead wire 16b connected to a power supply in a motor case 17. The shaft 16a of the electric motor 16 has, for example, a wheel attachment having an octagonal shape or other polygonal outer shape corresponding to the concave portion 13b of the driving side wheel 13, and the rotating motion of the rotating wheels 13, 14 can be achieved by inserting the shaft 16a into the concave portion 13b of the driving side wheel 13 via the wheel attachment.

Although not shown in detail, the motor case 17 can be provided with a rotation stop portion for preventing rotation of the motor body itself. Herein, a threaded portion that can be screwed with the wheel housing 12 of the drive wheel portion 9 is provided in the opening portion of the motor case 17, whereby the motor portion 10 is attached to the drive wheel portion 9. However, various connecting means such as one-touch attachment and detachment may be adopted.

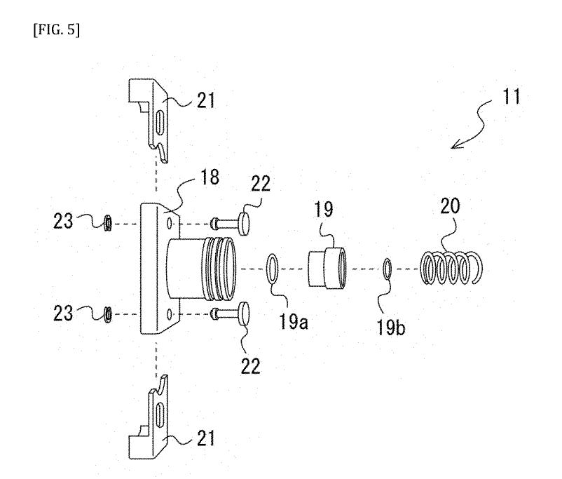

Further, the treatment tool driver 7 includes an endoscope connection portion 11 which enables the treatment tool driver 7 to be attached to the endoscope body. As shown in the exploded perspective view in FIG. 5, the endoscope connection portion 11 includes: a casing member 18 having a cylindrical portion provided in a central region on a surface of a plate-like portion; a sealing block 19 which has O-rings 19a, 19b at the respective inner end sides of the sealing block 19 and which is provided inside the cylindrical portion of the casing member 18 so as to be displaceable in the axial direction; a coil spring 20 disposed inside the sealing block 19; a pair of clamps 21 inserted from both sides of the casing member 18 into the casing member 18; and a pin 22 and an annular stopper 23 for supporting the pair of clamps 21, respectively, to the casing member 18.

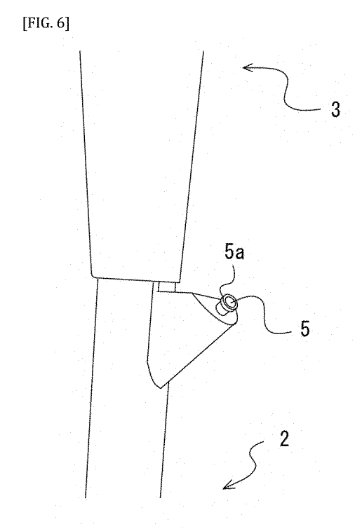

In the endoscope 1 of this embodiment, as shown in FIG. 6, the treatment tool insertion port 5 is provided with a tubular attaching protrusion 5a having a ring-shaped portion with a larger outer diameter at the distal end. In attaching the treatment tool driver 7 to the endoscope body, the attaching protrusion 5a of the endoscope body is fitted into a recessed portion provided on the back surface of the casing member 18 while spreading the pair of clamps 21 of the endoscope connecting portion 11 over both sides of the casing member 18.

Then, the attaching protrusion 5a is inserted into the recessed portion on the back surface of the casing member 18 while resisting a repulsive force of the coil spring 20 disposed in the sealing block 19, and at this time, by pushing the clamps 21 inward, a semicircular concave portion at the tip of each of the clamps 21 is hooked and engaged with a ring-shaped portion of the attaching protrusion 5a.

The endoscope connecting portion 11 is thus fixed to the treatment tool insertion port 5 of the endoscope body, so that the treatment tool driver 7 can be attached to the endoscope body.

On the other hand, when detaching the treatment tool driver 7 attached to the endoscope body from the endoscope body, the clamps 21 present inside are pulled out and moved. As a result, the engagement state of the distal end of the clamp 21 with the ring-shaped portion of the attaching protrusion 5a is released, so that the endoscope connection portion 11 is detached from the endoscope body under the assistance of the biasing force of the coil spring 20.

Here, the endoscope connecting portion 11 can be screwed and fixed to the drive wheel portion 9 by a screw or the like provided at the tip of the cylindrical portion of the casing member 18, for example.

By fixing the endoscope connecting portion 11 to the drive wheel portion 9 to attach the endoscope connecting portion 11 to the treatment tool insertion port 5 of the endoscope body, the endoscope connecting portion 11 is sandwiched between the drive wheel portion 9 and the treatment tool insertion port 5 to compress the O-rings 19a, 19b provided inside the sealing block 19, so that the O-rings 19a, 19b serve to prevent leakage of a liquid which may adhere to the treatment tool 51.

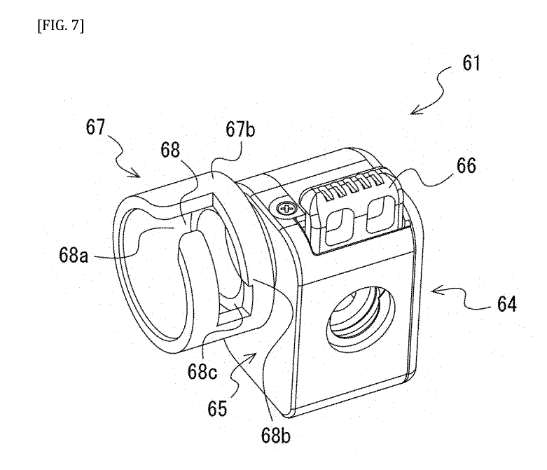

FIG. 7 shows a variation of a wheel housing 61 that can be used for the treatment tool driver. The wheel housing 61 is provided with an endoscope connecting portion. If the electric motor 16 is to be detached after once mounting the electric motor 16 so as to rotate the rotating wheel disposed therein, at least a part of the wheel housing 61 is destroyed and any reuse is prevented.

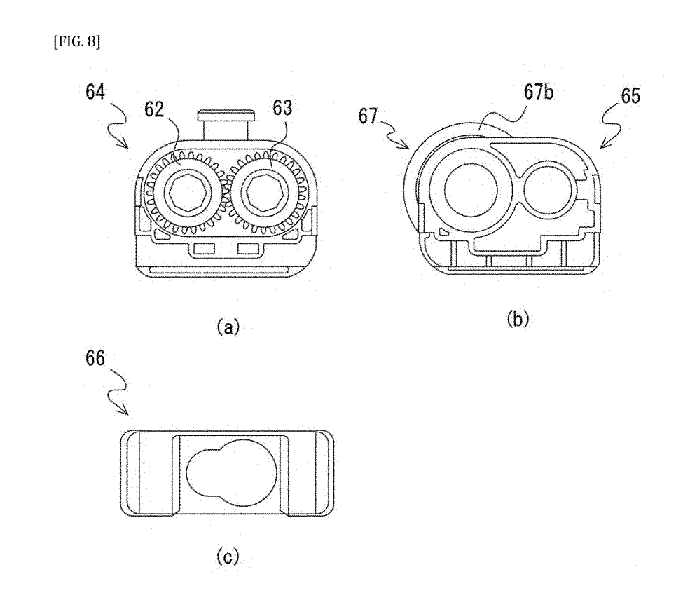

As shown in the exploded view in FIG. 8, the wheel housing 61 mainly includes: rotating wheels 62, 63 having substantially the same configuration as described above; a housing member 64 for housing the rotating wheels therein; and a cover member 65 attached to cover an opening portion of the housing member 64; and a substantially flat plate-like sliding lock member 66 which engages with the attaching protrusion 5a of the endoscope body and connects the wheel housing 61 to the endoscope body. Although the housing member 64 and the cover member 65 are connected by a screw in the illustrated example, it is preferable to connect them by ultrasonic waves, adhesives, or laser bonding, in terms of improvement of a production efficiency and airtightness.

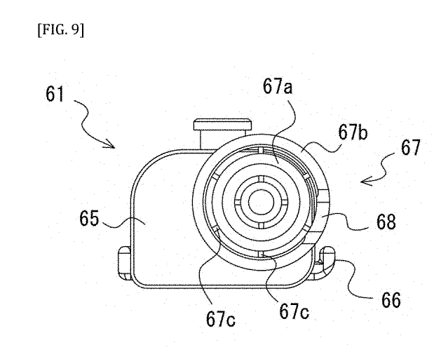

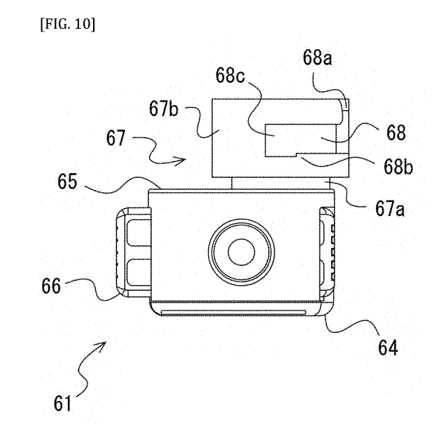

As shown in FIGS. 9 to 11, the outer surface of the cover member 65 of the wheel housing 61 has a tubular connecting portion 67 to which the electric motor 16 is attached. The tubular connecting portion 67 includes a cylindrical base portion 67a formed in the cover member 65; a distal end holding portion 67b which has a cylindrical shape with inner and outer diameters larger than the base portion 67a and around which a L-shaped slit 68a through which a convex portion 16c provided on the outer surface of the electric motor 16 is passed is formed to surround the periphery of a part of the electric motor 16 and hold it; and multiple, in the illustrated example, six easily breakable portion 67c provided at equal intervals in the circumferential direction, which extends in a radial direction to have a narrow rod shape between the base portion 67a and the distal end holding portion 67b to connect them with each other, as can be seen from FIG. 9. When attaching the electric motor 16, the slit 68 of the distal end holding portion 67b is fitted into the convex portion 16c provided on the outer surface of the electric motor 16, and an opening portion 68a is provided on the outer side of the slit 68, and a widened portion 68c is formed via a step 68b at the rearmost part of the slit 68. The slit 68 is in the form of a substantially L-shape extending from the opening portion 68a in the axial direction and then bent at a right angle in a substantially central region in the axial direction to extend along the circumferential direction.

When attaching the electric motor 16 to the tubular connecting portion 67, the electric motor 16 is firstly inserted into the distal end holding portion 67b of the tubular connecting portion 67. Herein, the electric motor 16 is inserted in the circumferential direction in which the convex portion 16c on the outer surface of the electric motor 16 coincides with the opening portion 68a of the slit 68 of the distal end holding portion 67b, while at the same time inserting the convex portion 16c of the electric motor 16 into the slit 68 from the opening portion 68a.

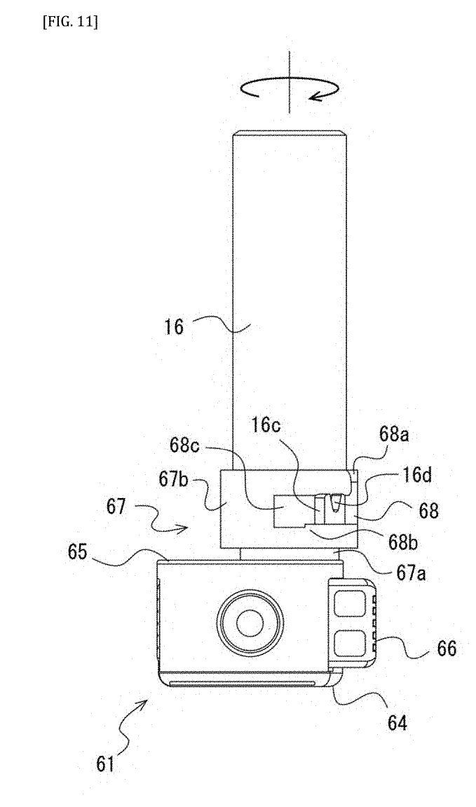

Then, as shown by the arrow in FIG. 11, the electric motor 16 is rotated around its central axis, and the convex portion 1c is moved deep inside the slit 68. The convex portion 16c then reaches the widened portion 68c through the step 68b in the slit 68, and the electric motor 16 is rotated until the convex portion 16c is fitted into the slit 68b, thereby completing the attachment.

After thus attaching the electric motor 16 to the tubular connecting portion 67, the convex portion 16c fitted in the widened portion 68c of the slit 68 prevents rotation in the direction of removing the electric motor 16 by the step 68b caught on the convex portion 16c. Nevertheless, if it is attempted to rotate the electric motor 16 with a stronger force, the easily breakable portion 67c between the base portion 67a of the tubular connection portion 67 and the distal end holding portion 67b will be broken or the step 68b will be scraped, so that the reattachment of the electric motor 16 to the tubular connecting portion 67 will not be possible. This is preferable in terms of hygiene because the wheel housing 61 is prevented from being repeatedly used.

In order to effectively fix the convex portion 16c within the widened portion 68c of the slit 68 after thus attaching the electric motor 16 to the tubular connecting portion 67, the convex portion 16c having a substantially square block shape in a plan view is preferably provided with a depressed portion 16d recessed substantially in a V-shape from the side surface located on the side opposite to the step 68b, as shown in the illustrated example.

In addition, the sliding lock member 66 can be traveled in the longitudinal direction between a locking position and a releasing position which are engaged with the attaching protrusion 5a of the endoscope body. However, in the illustrated example, as the sliding lock member 66 is repeatedly traveled, at least one side portion of the sliding lock member 66 is abraded at a contact portion(s) of the housing member 64 and/or the cover member 65, whereby a resistance force will be eliminated between the locking position and the releasing position, and no fixing will occur at the locking position. This can allow the attachment of the wheel housing 61 to the endoscope body to be limited to a predetermined number of times.

In the above description, while the treatment tool driver 7 has been described by way of an example of the mechanism for driving the treatment tool 51 by the geared rotating wheels 13, 14, 62, 63, various drive mechanisms can be used in place of that mechanism. It is also considered that the treatment tool driver 7 is attached to a position other than the treatment tool insertion port 5 of the endoscope body. However, by configuring the treatment tool driver to attach it to the treatment tool insertion port 5, the treatment tool driver 7 can be used without changing the structure of the existing endoscope.

In the example shown in FIG. 1, the drive controller 8 for controlling the forward movement and the backward movement of the treatment tool 51 by the treatment tool driver 7 is a foot pedal operated by the user's foot. In this case, it is possible to effectively utilize the feet of the user, which is often not used.

The drive controller 8 is provided with a switch as an input portion 24 for receiving an input from the outside; and a wiring as an output portion 25 for transmitting a signal corresponding to the input received by the input portion 24 to the treatment tool driver 7.

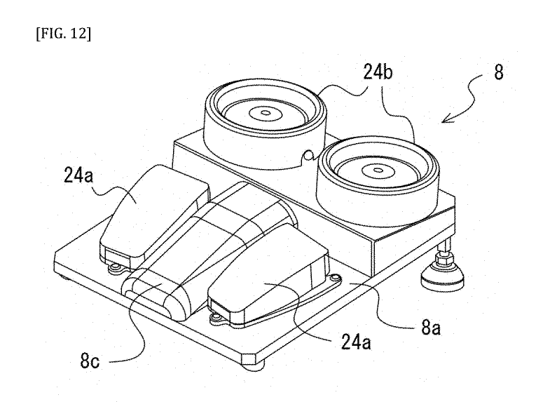

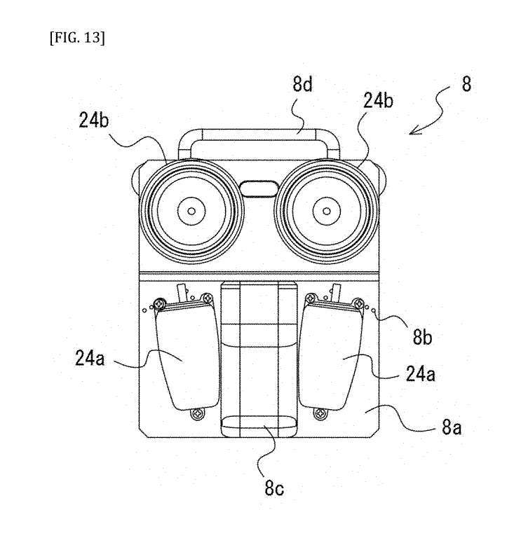

Further, in this case, the foot pedal as the drive controller 8 further includes, in addition to, as the input portion 24, a pair of low-speed switches for the forward/backward movement of the treatment tool 51, disposed on the substrate 8a, as shown in enlarged views of FIGS. 12 and 13, a pair of high-speed switches 24b for executing the forward/backward movement at a speed higher than the movement speed of the forward/backward movement by the low-speed switches 24a The foot pedal has total four switches.

Thus, the drive controller 8 can receive inputs of different speeds according to the intention of the user or the like, and in accordance with the received inputs, the output portion can transmit different signals of the movement speed of the forward/backward movement of the treatment tool to the treatment tool driver 7. The number of switches may be from one to three or five or more.

The high-speed switches 24b each having such a high movement speed can achieve, for example, shortening of the time taken from the start of the insertion of the treatment tool 51 into the treatment tool channel and the arriving of the tip portion of the treatment tool 51 at the distal end portion 2a of the insertion portion 2, or shortening of the time taken to remove completely the treatment tool 51 arranged in the treatment tool channel. On the other hand, the low-speed switches 24a each having a lower movement speed can be used for delicate operations such as a forward movement of the tip portion of the treatment tool 51 to a diseased part to be treated or a backward movement to the diseased part after the tip portion of the treatment tool 51 reaches the distal end portion 2a of the insertion portion 2.

In addition, the drive controller 8 preferably includes a motor connecting connector and a power supply (not shown), as well as a computer communication port (also not shown) for allowing fine adjustment or change of each movement speed of a plurality of stages by using an electric computer or the like, on the back side (the upper side in FIG. 13) of the support 8a. It is also possible to connect the drive controller 8 to an electronic computer so that motor drive data can be edited. The setting using such an electronic computer can also allow assignment of the above four switches 24a, 24b to be changed, and three or more of the switches 24a, 24b for a higher speed or a lower speed having different speeds to be used. It should be noted that the communication between the electronic computer and the drive controller 8 can also be a wireless communication based on a short-distance wireless communication standard or the like.

In this case, it is preferable to provide a treatment tool detection sensor or a stopper for automatically lowering or stopping the speed when the tip portion of the treatment tool 51 approaches the distal end portion 2a of the insertion portion 2.

For example, a stopping sensor (an optical sensor, a magnetic sensor) used for stopping a high-speed forward movement of the treatment tool 51 can be provided inside or around the treatment tool driver 7. As will be described below, when the treatment tool driver is integrally incorporated in the endoscope body, the stopping sensor can be built in the endoscope body. Based on this, a stopping marking for the high-speed forward movement of the treatment tool 51 (for example, a black colored mark or a silver colored mark or a mark with a concave and convex shape) is applied onto a part on a base side than a stopped position detected by the stopping sensor or over the entire stopped position.

This can allow the stopping mark applied on the treatment tool 51 to be detected by the above stopping sensor and the high-speed forward movement of the treatment tool 51 to be automatically stopped, when automatically inserting the treatment tool 51 into the endoscope 1 at a high speed, during replacement for insertion of the treatment tool 51. Alternatively, even if the switch for the high-speed forward movement is mistakenly pushed in a state where the treatment tool 51 is near the distal end of the endoscope 1 or in a state where the treatment tool 51 is led out from the distal end of the endoscope 1 (which is a situation where it is not necessary to move the treatment tool 51 forward at a high speed), the stopping sensor always detects the stopping mark, so that any high-speed forward movement of the treatment tool 51 can be prevented. The function of automatically stopping the high-speed forward movement of the treatment tool 51 is extremely effective for preventing accidents such as piercing and drilling of the treatment tool 51 into the digestive tract wall or the like, and for safely using it.

It is also possible to control automatically the speed or the stop based on a period of time from the start of insertion of the treatment tool 51 using a relationship between the rotation speed of the rotation wheels 13, 14 and the forward movement distance of the treatment tool 51. Alternatively, the distance for inserting the treatment tool 51 has been determined according to a pattern of a length of the insertion portion 2 of the endoscope 1 to be used, and the rotation wheels 13, 14 has been allowed to rotate for the specific time according to the distance, whereby the treatment tool 51 can be inserted and automatically stopped at the high movement speed only by inputting the high-speed switch 24b once. In this case, a changeover switch for inputting pattern information of the length of the endoscope 1 to be used may be provided at any position of the drive controller 8 or the like.

The substrate 8 of the drive controller 8 may be provided with a plurality of holes 8b through which screws for attaching the low-speed switches 24b to the substrate 8, so as to be able to change an angle of the low speed switches 24a relative to the up-down direction or the right-left direction in FIG. 13 on the substrate 8a, according to the arrangement of the foot of the user.

Also, in the illustrated drive controller 8, a footrest portion 8c on which a user's foot can be placed is inserted between a pair of low speed switches 24a which cause each operation of the forward movement and the backward movement of the treatment tool 51.

The back side of the support 8 is provided with a handle 8d that can be grasped when carrying the drive controller 8, and the bottom side of the support is provided with an electrical equipment box (not shown).

Further, preferably, the drive controller detects that the user inserts the treatment tool 51 into the drive wheel portion 9 of the treatment tool driver 7 at a speed higher than a predetermined speed, and in this case, the drive controller controls that the treatment tool 51 is rotated at a high speed, and/or, the drive controller detects that the user or the like pulls the treatment tool 51 in the direction of withdrawing the treatment tool 51 from the drive wheel portion 9 at a speed higher than a predetermined speed, and in this case, the drive controller controls that the treatment tool 51 is moved backward at a high speed. This can allow the number of switches of the drive controller to be reduced by eliminating the high-speed switches 24b as described above, thereby preventing an operation due to an error of the switches in use and also reducing production costs for the drive controller.

It is to be noted that in the insertion of the treatment tool 51 such as a forceps to the middle of the insertion portion 2 of the endoscope 1, when a power failure occurs or a signal cable incorrectly connected to the power cable or the motor is pulled out and the cable is then inserted again before pressing the high-speed switches 24b, the forceps may jump out from the distal end portion 2a of the insertion portion 2 of the endoscope 1 at a high speed. In such a case, the diseased part of the patient may be damaged. In order to prevent this, when the power is connected, preferably, the rotation of the motor is electrically controlled such that the rollers are rotated in the rotation direction of withdrawing the treatment tool 51 such as the forceps for a predetermined time.

It is also preferable to control the rollers to rotate not only at the time of occurrence of a power outage or the like but also during normal use in the rotation direction of withdrawing the treatment tool 51 at the time of power source connection. This can allow the operation confirmation at a preparation stage (more specifically confirmation that the cables are correctly connected or the electronic boards, parts or the like are not broken before use).

The drive controller may be a drive control means (not shown) for controlling the operation of the treatment tool driver 7 based on not only the above-mentioned foot pedal but also on a part or the whole of the body such a hand(s) and arm(s), other than the foot, of an object person such as a user or an assistant, in addition to the foot pedal as described above.

Specifically, it may be various switches, as well as sensors for eyes, motion, gesture and a voice, specifically an acceleration sensor for detecting movement in each axis direction, a gyro sensor for detecting angular velocity, electricity, magnetism or light, a sensor for sensing the movement of the subject using ultrasonic waves or the like, or the like.

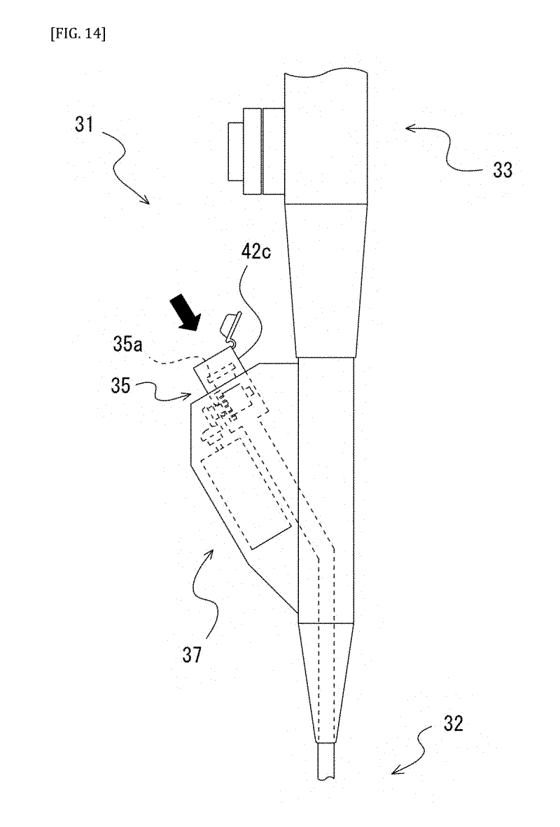

FIG. 14 shows an endoscope 31 according to other embodiment of the present invention. The endoscope 31 of the other embodiment shown in FIG. 14 has substantially the same structure as that of the embodiment shown in FIG. 1, with the exception that a treatment driver 37 is integrally incorporated into the endoscope body including the insertion portion 32 and the operation portion 33.

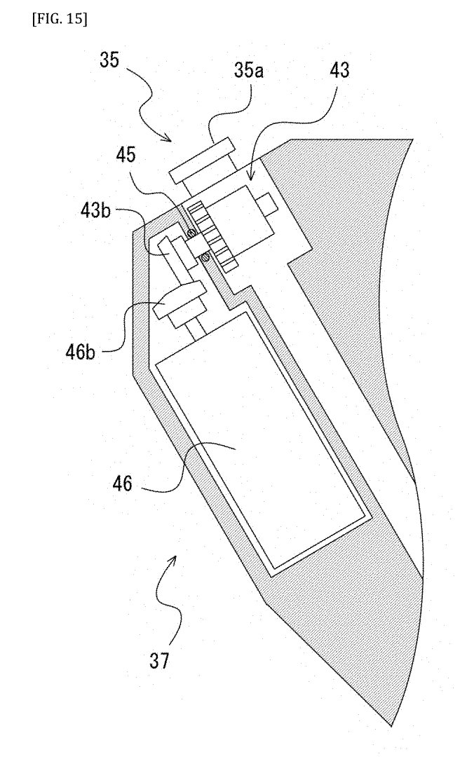

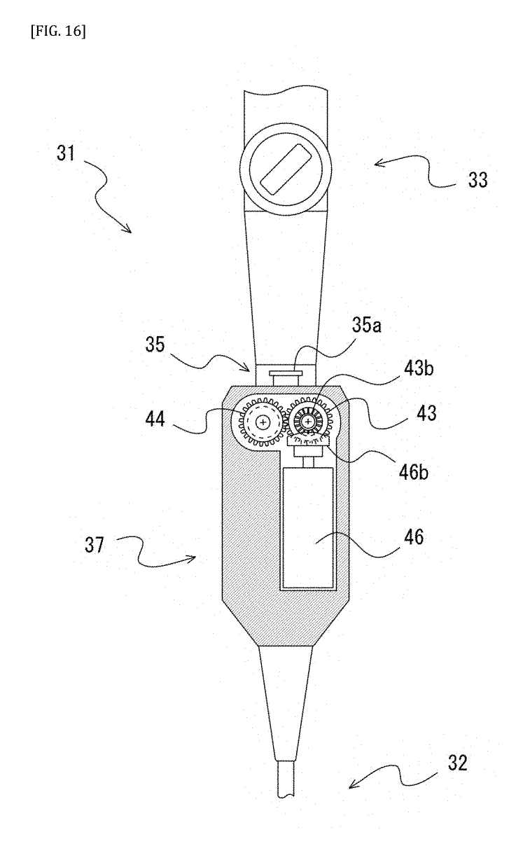

More specifically, as shown in FIGS. 15 and 16, the endoscope 31 shown in FIG. 14 includes, in the inside of the treatment tool insertion port 35 between the insertion portion 32 and the operation portion 33, a pair of rotating wheels as a driving side wheel 43 and a driven side wheel 44 each having substantially the same configuration as that of the endoscope 1; and a treatment tool driver 37 including an electric motor 46 for rotatingly driving the driving side wheel 43 by supplying electric power from a power source (not shown). As shown in FIG. 15, a sealing material 45 for preventing the liquid adhering to the treatment tool from flowing in the motor 46 side may be provided between the rotating wheels 43, 44 and the motor 46.

In the endoscope 31, the driving side wheel 43 and the electric motor 46 are arranged inside the treatment tool insertion port 35 in such a manner that their rotation axes form a predetermined angle. Therefore, a pair of bevel gears 43b, 46b are provided at the connecting position between the wheel 43 and the electric motor 46 so as to enable transmission of the power between the rotation axes at which the angle are formed.

The inside of the treatment tool driving means 37 is in communication with the treatment tool channel extending from the treatment tool insertion port 35 to the insertion portion 32. Here, as shown in FIG. 14, an insertion port cap 42c is attached to an attaching protrusion 35a of the treatment tool inserting port 35.

However, the specific structure and arrangement position of the treatment tool driver 37 are not limited to those shown in the figure.

In order to insert the treatment tool into the endoscope 31, as shown by the arrow in FIG. 14, the treatment tool is moved from its distal end portion through the insertion port cap 42c of the treatment tool insertion port 35 to the treatment tool driver 37. Once the distal end portion of the treatment tool arrives between the rotating wheels 43, 44 of the treatment tool driver 37, for example, the same driving controller as that described above is operated to rotate the electric motor 46, thereby rotatively driving the rotation wheels 43, 44, so that the treatment tool whose distal end portion is disposed between the rotating wheel 43, 44 can be forced to move forward by the rotation wheels 43, 44 and guided by the treatment tool channel to allow the distal end portion of the treatment tool to reach the distal end portion of the insertion portion 32. When the electric motor 46 is rotated in the reverse direction, the treatment tool can be moved backward, and the treatment tool can be removed from the insertion portion 32.

Such an endoscope 31 does not require the attachment and detachment operations of the treatment tool driver 7 required for the endoscope 1 described above as shown in FIG. 1. Therefore, further improvement of convenience can be achieved. Further, since the treatment tool driver 7 is incorporated into the endoscope body, the endoscope 31 can be downsized as a whole.

### DESCRIPTION OF REFERENCE NUMERALS

1, 31 endoscope
2, 32 insertion portion
2a distal end portion of insertion portion
2b curved portion of the insertion portion
3, 33 operation portion
4 connecting portion
5, 35 treatment tool insertion port
5a, 35a attaching protrusion
6 angle knob
7, 37 treatment tool driver
8 drive controller
8a substrate
8b hole
8c footrest portion
8d handle
9 drive wheel portion
10 motor portion
11 endoscope connecting portion
12, 61 wheel housing
12a treatment tool inlet
12b treatment tool outlet
12c, 42c insertion port cap
13, 14, 43, 44, 62, 63 rotation wheel (driving side wheel, driven side wheel)
13a, 14a roller
13b concave portion
15, 45 sealing material
16, 46 electric motor
16a shaft
16b lead wire
16c convex portion
16d depressed portion
17 motor case
18 casing member
19 sealing block
19a, 19b O ring
20 coil spring
21 clamp
22 pin
23 stopper
24 input portion
24a low-speed switch
24b high-speed switch
25 output portion
43b, 46b bevel gear
51 treatment tool
64 housing member
65 cover member
66 sliding locking member
67 tubular connecting portion
67a base portion
67b distal end holding portion
67c easily breakable portion
68 slit
68a opening portion
68b step
68c widened portion

1. An endoscope comprising: a tubular insertion portion to be inserted into a living body; and one or more treatment tool channels formed inside the insertion portion, the treatment tool channels being configured to guide a treatment tool by passing the treatment tool through the treatment tool channel, wherein the endoscope comprises:
a treatment tool driver for allowing a forward movement and/or a backward movement of the treatment tool in the treatment tool channel; and
a drive controller for controlling the forward movement and/or the backward movement of the treatment tool driven by the treatment driver.

2. The endoscope according claim 1, wherein the endoscope comprises an endoscope body including the insertion portion, and wherein the treatment tool driver comprises a treatment tool drive module attachable to and detachable from the endoscope body.

3. The endoscope according to claim 1, wherein the endoscope comprises an endoscope body including the insertion portion, and wherein the treatment tool driver is integrally incorporated in the endoscope body.

4. The endoscope according to claim 1, wherein the treatment tool driver comprises: one or more rotating wheels that are brought into contact with the treatment tool and allow the forward movement and/or backward movement of the treatment tool; and a motor for driving the rotating wheels to rotate them.

5. The endoscope according to claim 4, wherein the treatment tool driver further comprises a wheel housing for containing the rotating wheels, and wherein the motor is attached to the wheel housing and at least a part of the wheel housing is destroyed by removing the motor.

6. The endoscope according to claim 4, wherein the rotating wheels are a pair of rotating wheels that are rotated in opposite directions and allow the forward movement and/or backward movement of the treatment tool sandwiched between the rotating wheels

7. The endoscope according to claim 1, wherein the drive controller comprises: an input portion for receiving an input from the outside; and an output portion for transmitting a signal corresponding to the input received by the input portion to the treatment tool driver.

8. The endoscope according to claim 7, wherein the input portion of the drive controller is configured to be capable of receiving a plurality of types of inputs, and the output portion is configured to generate signals having different movement speeds of the forward movement and/or the backward movement of the treatment tool driven by the treatment tool driver, in response to the input received by the input portion.

9. The endoscope according to claim 1, wherein the drive controller is a foot pedal manipulated by a user.

10. The endoscope according to claims claim 1, comprising a sensor capable of detecting a marking made in a predetermined position of the treatment tool.

11. A treatment tool drive module, the treatment tool drive module being used for an endoscope comprising an endoscope body including a tubular insertion portion to be inserted into a living body, the treatment tool drive module being attachable to and detachable from the endoscope body, and the treatment tool drive module allowing a forward movement and/or a backward movement of the treatment tool in a treatment tool channel formed inside the insertion portion of the endoscope body.

12. The treatment tool drive module according to claim 11, wherein the treatment tool drive module comprises: one or more rotating wheels that are brought into contact with the treatment tool and allow the forward movement and/or backward movement of the treatment tool; and a motor for driving the rotating wheels to rotate them.

13. The treatment tool drive module according to claim 12, further comprising a wheel housing for containing the rotating wheels, and wherein the motor is attached to the wheel housing and at least a part of the wheel housing is destroyed by removing the motor.

14. The treatment tool drive module according to claim 12, wherein the rotating wheels are a pair of rotating wheels that are rotated in opposite directions and allow the forward movement and/or backward movement of the treatment tool sandwiched between the rotating wheels.

15. The treatment tool drive module according to claim 11, comprising a sensor capable of detecting a marking made in a predetermined position of the treatment tool.
